(19) United States
(12) Patent Application Publication
(10) Pub. No.: US 2025/0366786 A1
(43) Pub. Date: Dec. 4, 2025

(54) Health Monitoring Diaper System with Integrated Multi-Sensor Technology and Real-Time Alert Capabilities

(71) Applicant: Zainab Zaki Alkhamis, Abu dhabi (AE)

(72) Inventor: Zainab Zaki Alkhamis, Abu dhabi (AE)

(21) Appl. No.: 18/676,640

(22) Filed: May 29, 2024

(51) Int. Cl. A61B 5/00; A61B 5/0205; A61B 5/1455; A61F 13/42; A61F 13/84

(52) U.S. Cl. CPC A61B 5/6808; A61B 5/0022; A61B 5/0205; A61B 5/14552; A61B 5/7405; A61B 5/746; A61F 13/42; A61F 2013/424; A61F 2013/427; A61F 2013/8473; A61F 2013/8488

(57) ABSTRACT

The present invention discloses an intelligent diaper monitoring device comprising a housing equipped with an attachment mechanism for detachable securing to a diaper. The housing includes a power source, a sensor array, a wireless communication module, and a controller. The sensor array features a wetness detection circuit, a humidity sensor, an ionic composition sensor, and one or more physiological sensors for monitoring at least one of heart rate, oxygen saturation, and temperature. The controller processes signals from the sensor array and communicates them to an external device via the wireless communication module. Additionally, the system includes a Wi-Fi connected hub or relay in communication with the wireless communication module, comprising an advanced wireless communication module, an LED, and a speaker to provide visual and auditory alarms, and to facilitate robust data transmission to external devices. This device provides comprehensive monitoring and real-time data transmission to ensure effective diaper management and wearer health monitoring.

### FIELD OF INVENTION

The present invention relates generally to a comprehensive diaper management system incorporating advanced multi-sensor technology for real-time monitoring of health parameters and environmental conditions, designed to provide timely alerts and improve caregiving for infants, elderly individuals, and pets.

### BACKGROUND

In recent years, advancements in caregiving technology have aimed to address the persistent challenges associated with diaper management for infants, elderly individuals, and pets. Traditional diaper systems primarily rely on periodic checks and manual monitoring to assess the need for changes, often leading to delays that can result in discomfort, skin irritation, and other health issues. This approach is not only inefficient but also inadequate for providing the real-time, comprehensive care that modern caregiving demands.

Current systems on the market, while incorporating basic wetness detection, fall short in offering a holistic solution that integrates multiple health parameters. They typically use single-function sensors that detect moisture, which only addresses one aspect of the problem. These systems lack the sophistication to monitor additional crucial indicators such as temperature, electrolyte levels, and other physiological parameters. This limitation restricts their ability to provide a full picture of the user's health and environmental conditions, often leading to reactive rather than proactive care.

One example of an existing system that goes some way towards addressing these needs is disclosed in the inventor's existing patent publication, U.S. Pat. No. 9,675,496 B1, which discloses a diaper change alert system, method, and computer-readable medium a diaper with wetness detection circuit installed inside a seat portion of the diaper, an ionic composition sensor installed inside the seat portion of the diaper, a humidity sensor installed inside the seat portion of the diaper, processing circuitry. The processing circuitry is configured to compute number of waste cycles per diaper change based on a signal received from the wetness detection circuit, compute a total time per diaper change based on a signal received from the wetness detection circuit, compute a rash threshold using rash information, receive sensor data, determine whether the rash threshold is reached, and transmit, via a network, a diaper change alert to an external device.

Moreover, existing products frequently involve cumbersome setups with multiple components that are not always user-friendly or adaptable to different environments. The integration of these systems into everyday caregiving routines can be complex and time-consuming, requiring caregivers to manage multiple devices and interfaces. Additionally, the data provided by these systems is often fragmented, necessitating manual interpretation and decision-making, which increases the burden on caregivers and leaves room for human error.

The need for a more advanced and integrated approach has become evident, especially in settings like nurseries, elderly care facilities, and homes with special needs individuals. These environments require a solution that not only monitors wetness but also provides insights into broader health metrics. Furthermore, there is a growing demand for systems that can seamlessly connect to digital health platforms, allowing for real-time data sharing and remote monitoring by healthcare professionals. The ideal solution would be one that is not only easy to use but also capable of delivering actionable insights that enable timely interventions and improve overall health outcomes.

In response to these challenges, there has been a significant push towards developing intelligent systems that leverage advanced sensor technologies and wireless connectivity. These innovations aim to create a comprehensive caregiving tool that addresses the limitations of current products and sets a new standard in diaper management. By incorporating a range of sensors to monitor various health indicators and integrating with mobile applications for real-time alerts and data analysis, these systems promise to transform how caregivers manage diaper-related care. This evolution in technology is poised to provide a more efficient, effective, and user-friendly solution that meets the complex needs of modern caregiving.

It is within this context that the present invention is provided.

### SUMMARY

The present invention relates to an intelligent diaper monitoring device that includes a housing with an attachment mechanism for detachably securing the device to a diaper. The housing contains a power source, a sensor array, a wireless communication module, and a controller. The sensor array comprises a wetness detection circuit, a humidity sensor, an ionic composition sensor, and one or more physiological sensors for monitoring at least one of heart rate, oxygen saturation, pH levels, and temperature. The controller processes signals from the sensor array and communicates them to an external device via the wireless communication module.

Additionally, the system includes a Wi-Fi connected hub or relay that acts as an intermediary between the sensor device and the external devices, featuring an advanced wireless communication module, an LED, and a speaker to provide visual and auditory alarms, and to facilitate robust data transmission.

In some embodiments, the wetness detection circuit within the sensor array includes a moisture sensor and a signal processor. The moisture sensor detects moisture levels within the diaper, and the signal processor processes these signals. This configuration allows for accurate and timely detection of wetness, ensuring that caregivers are promptly informed when a diaper change is needed.

In some embodiments, the moisture sensor comprises a pair of conductive traces disposed on an absorbent layer of the diaper. This design enhances the sensor's ability to detect even minimal levels of moisture, providing a reliable indicator of the diaper's condition.

In some embodiments, the ionic composition sensor within the sensor array includes an ion-selective electrode array configured to detect specific ions such as sodium, potassium, calcium, and chloride. This capability allows for the monitoring of various health indicators present in the moisture, providing valuable data on the wearer's physiological state.

In some embodiments, the device includes a pressure sensor within the sensor array, configured to detect the position or movements of the wearer. This feature provides additional information on the wearer's activity and comfort levels, aiding in comprehensive monitoring.

In some embodiments, the wireless communication module includes a Bluetooth transceiver for short-range communication with a user device. This enables easy and efficient data transfer to the caregiver's smartphone or other user device.

In some embodiments, the device further includes a Wi-Fi transceiver for long-range communication with a remote server or network. This feature supports continuous monitoring and data logging, allowing for remote access and analysis of the wearer's condition.

In some embodiments, the housing includes an indicator light configured to provide a visual status of the device's connectivity and operation. This provides caregivers with an immediate and clear understanding of the device's status.

In some embodiments, the power source within the housing is a rechargeable battery. This ensures that the device can be used for extended periods without the need for frequent battery replacements, enhancing convenience for caregivers.

In some embodiments, the attachment mechanism is configured to securely attach the housing to the exterior of a diaper. This ensures that the device remains in place during use, providing consistent monitoring without causing discomfort to the wearer.

In some embodiments, the housing is waterproof to protect the internal components from moisture exposure. This durability ensures the longevity and reliability of the device even in a moist environment.

In some embodiments, the physiological sensors within the sensor array include a pulse oximeter sensor for measuring oxygen saturation levels. This provides critical data on the wearer's respiratory function, contributing to comprehensive health monitoring.

In some embodiments, the device includes a temperature sensor within the sensor array, configured to monitor the ambient temperature within the diaper. This feature helps in maintaining optimal comfort for the wearer by alerting caregivers to any significant changes in temperature.

In some embodiments, the wireless communication module is configured to transmit data to a dedicated mobile application installed on a user device. This integration allows caregivers to receive real-time alerts and access historical data analysis, facilitating informed decision-making.

In some embodiments, the device further comprises a detachable layer made of soft material that hosts the wetness detection circuit, humidity sensor, and ionic composition sensor, and is attachable to a conventional diaper. This feature allows the advanced monitoring capabilities to be used with standard diapers, offering flexibility and convenience to caregivers.

### BRIEF DESCRIPTION OF THE DRAWINGS

Various embodiments of the invention are disclosed in the following detailed description and accompanying drawings.

FIG. 1 illustrates an example intelligent diaper monitoring system comprising a user's smartphone, a Wi-Fi connected hub, and a sensor device attached to a diaper, showing the communication between components.

FIG. 2 illustrates an example block diagram of the sensor device, detailing its internal components including the power source, sensor array, controller, and wireless communication module.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The term “housing” as used herein refers to any structure or casing that encloses and protects the internal components of the intelligent diaper monitoring device. The housing may be made of plastic, silicone, or other durable materials suitable for withstanding the conditions of use. It may be designed to be waterproof or water-resistant to protect the internal components from moisture and other environmental factors.

The term “attachment mechanism” refers to any means by which the housing is secured to a diaper. This can include, but is not limited to, adhesive strips, Velcro, snap buttons, clips, or any other fastening method that ensures secure and detachable attachment to a diaper.

The term “power source” encompasses any device or system that provides electrical energy to the components of the intelligent diaper monitoring device. This includes, but is not limited to, rechargeable batteries, disposable batteries, solar cells, or any other suitable power supply. Example implementations could be a lithium-ion rechargeable battery or an alkaline disposable battery.

The term “sensor array” includes various sensors integrated within the housing to monitor different conditions. These sensors may include, but are not limited to:
Wetness Detection Circuit: A circuit comprising sensors such as conductive traces or moisture sensors that detect the presence and level of wetness.
Humidity Sensor: A sensor that measures the humidity levels within the diaper environment.
Ionic Composition Sensor: A sensor capable of detecting specific ions such as sodium, potassium, calcium, and chloride in the moisture present in the diaper, potentially using an ion-selective electrode array.
Physiological Sensors: Sensors that monitor physiological parameters such as heart rate, oxygen saturation (e.g., using a pulse oximeter), pH levels, and temperature.

The term “wireless communication module” refers to any device or system that enables wireless data transmission from the intelligent diaper monitoring device to external devices. This may include, but is not limited to, Bluetooth transceivers, Wi-Fi transceivers, or any other wireless communication protocols. Example implementations include Bluetooth Low Energy (BLE) for short-range communication with a smartphone or Wi-Fi for long-range communication with a remote server or network.

The term “controller” refers to the processing unit within the housing that receives signals from the sensor array, processes these signals, and communicates the processed data via the wireless communication module. The controller may be a microcontroller, a microprocessor, or any other suitable processing unit capable of performing these functions.

The term “external device” encompasses any device that receives data from the intelligent diaper monitoring device via the wireless communication module. This includes, but is not limited to, smartphones, tablets, computers, or any other electronic devices capable of receiving and displaying the transmitted data. Example external devices may include an iPhone running a dedicated mobile application or a cloud server that stores and analyzes the data for remote monitoring, research, and other applications.

### DESCRIPTION OF DRAWINGS

The present invention relates to an intelligent diaper monitoring device comprises a housing that can be securely attached to a diaper, containing a power source, a sensor array, a wireless communication module, and a controller. The sensor array includes a wetness detection circuit, a humidity sensor, an ionic composition sensor, and one or more physiological sensors for monitoring heart rate, oxygen saturation, and temperature. These sensors continuously collect data on the diaper's condition and the wearer's health, which is processed by the controller and transmitted wirelessly to external devices such as smartphones or remote servers.

In some embodiments, the system further includes a Wi-Fi connected hub or relay, which acts as an intermediary between the sensor device attached to the diaper and the user's external devices. This hub is equipped with an advanced wireless communication module to facilitate robust and long-range data transmission. Additionally, the hub features an LED and a speaker to create effective visual and auditory alarms, ensuring caregivers are promptly alerted to any critical conditions.

The primary benefit of this invention is its ability to provide caregivers with real-time alerts and detailed insights into the wearer's needs, enabling timely interventions and improving overall care. The wireless communication module ensures that data is readily accessible, allowing caregivers to monitor the wearer's condition remotely and efficiently. The inclusion of a Wi-Fi connected hub enhances the system's reliability and expands its communication range, making it suitable for larger environments such as homes, nurseries, and elderly care facilities.

Additionally, the device's design ensures that it is easy to attach to any standard diaper, making it versatile and convenient for a wide range of users. By integrating multiple sensors into a single, compact device, this invention offers a comprehensive solution that enhances the safety, comfort, and well-being of the wearer. The intelligent diaper monitoring device not only detects wetness but also monitors critical health indicators, providing a holistic approach to diaper management.

This innovation represents a significant advancement in caregiving technology, addressing the limitations of current systems and setting a new standard for diaper monitoring. The addition of a Wi-Fi connected hub or relay with advanced communication capabilities and effective alert mechanisms further solidifies the invention's role as a crucial tool in modern caregiving.

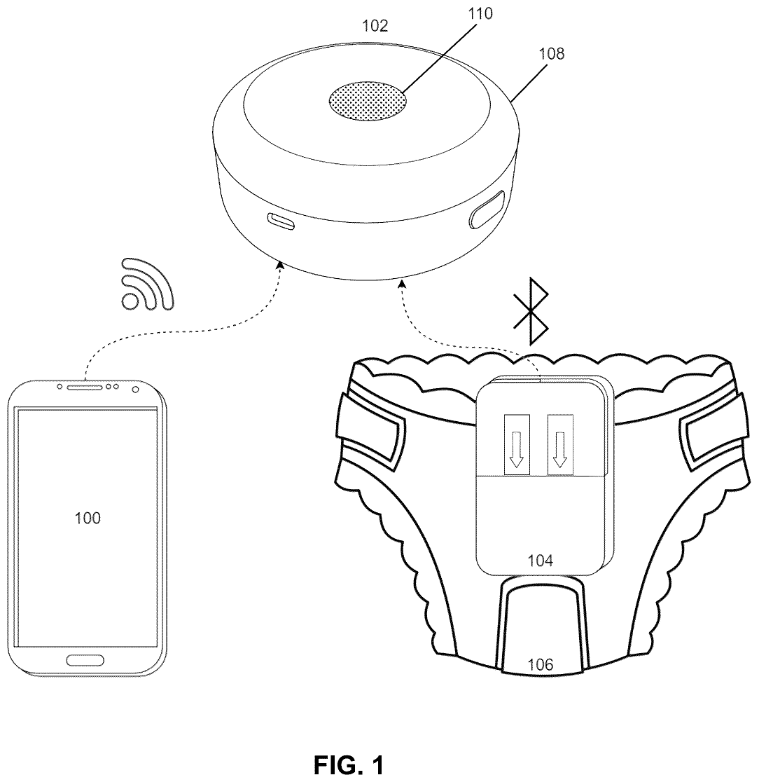

FIG. 1 illustrates the intelligent diaper monitoring system, which includes a user's smartphone 100, a Wi-Fi connected hub 102, and a sensor device 104 attached to an incontinent person's diaper 106. The smartphone 100 communicates with the hub 102 via Wi-Fi, while the hub 102 communicates with the sensor device 104 via Bluetooth.

The smartphone 100 is equipped with a dedicated mobile application designed to receive and display data transmitted from the hub 102. The hub 102 contains an advanced wireless communication module, an LED 108 for visual alerts, and a speaker 110 for auditory alarms. The sensor device 104, attached to the diaper 106, comprises a housing that encloses various sensors including a wetness detection circuit, a humidity sensor, an ionic composition sensor, and physiological sensors.

In a typical usage scenario, the sensor device 104 continuously monitors various parameters. For instance, the wetness detection circuit detects moisture levels within the diaper 106, and the physiological sensors measure heart rate and oxygen saturation. If the moisture sensor detects a moisture level that exceeds a predetermined threshold, and simultaneously, the humidity sensor detects high humidity indicative of prolonged wetness, the sensor device 104 processes these signals and sends an alert to the hub 102 via Bluetooth.

Upon receiving the alert from the sensor device 104, the hub 102 activates its LED 108 to flash and its speaker 110 to emit an alarm sound, providing immediate visual and auditory cues to caregivers in the vicinity. Simultaneously, the hub 102 transmits the data to the smartphone 100 over Wi-Fi. The mobile application on the smartphone 100 then displays a notification indicating the specific issues detected, such as “High moisture detected” and “Prolonged humidity,” allowing the caregiver to take prompt action to change the diaper 106 and address any potential health concerns.

This configuration ensures robust and effective communication between the components, providing real-time monitoring and alerts to enhance caregiving for infants, elderly individuals, and pets.

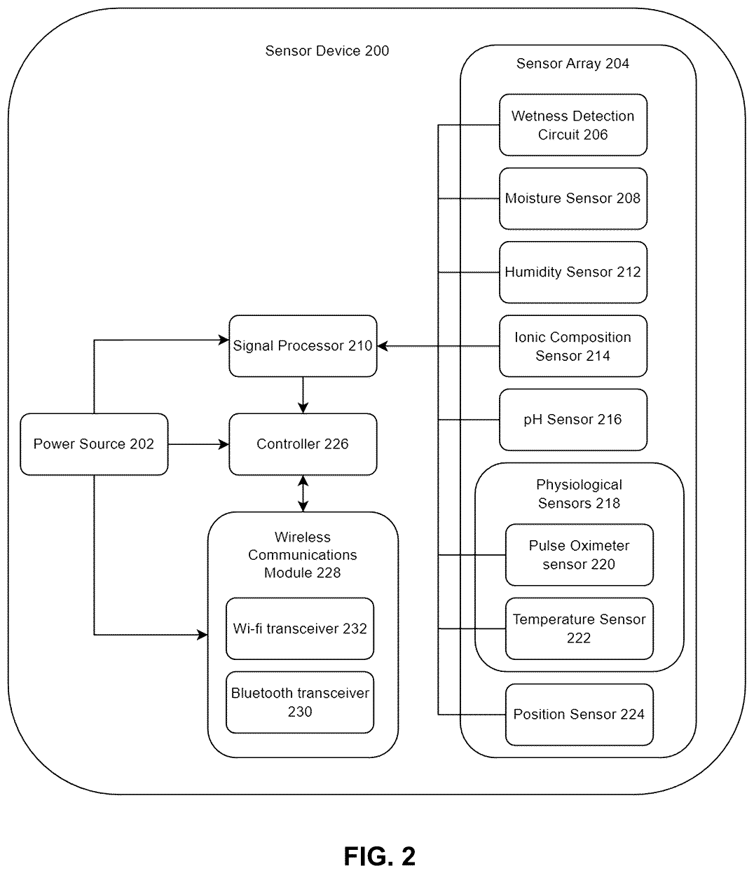

FIG. 2 illustrates a block diagram of the sensor device 200.

The sensor device 200 comprises various components integrated within a housing designed to be attachable to a diaper. The power source 202 supplies electrical energy to all the components within the sensor device 200. The power source 202 can be a rechargeable battery or another suitable power supply, ensuring continuous operation of the device.

The sensor array 204 includes multiple sensors for monitoring various parameters. The wetness detection circuit 206 detects moisture levels within the diaper. This circuit comprises a moisture sensor 208, which includes a pair of conductive traces, and a signal processor 210 that processes the signals from the moisture sensor 208.

The humidity sensor 212 measures the humidity levels within the diaper environment, providing data on the moisture conditions that may affect the wearer's comfort. The ionic composition sensor 214 detects specific ions such as sodium, potassium, calcium, and chloride in the moisture present in the diaper. This sensor may utilize an ion-selective electrode array to measure the ionic composition accurately. The physiological sensors 218 monitor the wearer's health parameters, including heart rate, oxygen saturation, pH levels, and temperature. The array includes a pH sensor 216, a pulse oximeter sensor 220 for measuring oxygen saturation and a temperature sensor 222 for detecting the ambient temperature within the diaper. The position sensor 224 detects the position or movement of the wearer, providing data on whether the wearer has been in the same position for too long, which could indicate the need for repositioning.

The controller 226 serves as the central processing unit of the sensor device 200. It receives signals from the sensor array 204, processes the data, and determines whether any of the monitored parameters exceed predetermined thresholds.

The wireless communication module 228 is responsible for transmitting the processed data from the controller 226 to the external devices. This module includes both a Bluetooth transceiver 230 for short-range communication with the Wi-Fi connected hub and a Wi-Fi transceiver 232 for potential direct long-range communication.

In operation, when the sensors in the sensor array 204 detect values exceeding the predetermined thresholds, the controller 226 processes these signals and sends an alert to the wireless communication module 228. The Bluetooth transceiver 230 transmits the data to the Wi-Fi connected hub, which can then alert caregivers via visual and auditory signals, and also transmit the data to a smartphone or other external devices for further action.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the intelligent diaper monitoring device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An intelligent diaper monitoring device comprising:
a housing comprising an attachment mechanism for detachably securing the system to a diaper;
a power source disposed within the housing;
a sensor array comprising:
a wetness detection circuit disposed within the housing;
a humidity sensor disposed within the housing;
an ionic composition sensor disposed within the housing;
one or more physiological sensors disposed within the housing, wherein said physiological sensors include sensors for monitoring at least one of heart rate, oxygen saturation, pH levels, and temperature;
a wireless communication module disposed within the housing for transmitting data;
a controller, the controller being configured to process signals received from the sensor array and communicate them to an external device via the wireless communication module;
a Wi-Fi connected hub or relay in communication with the wireless communication module, said hub comprising an advanced wireless communication module, an LED, and a speaker, the hub being configured to receive data from the wireless communication module and transmit it to the external device, as well as provide visual and auditory alarms.

2. The intelligent diaper monitoring device of claim 1, wherein the wetness detection circuit within the sensor array comprises: a moisture sensor configured to detect moisture levels within the diaper; a signal processor configured to receive and process signals from the moisture sensor.

3. The intelligent diaper monitoring device of claim 2, wherein the moisture sensor comprises: a pair of conductive traces disposed on an absorbent layer of the diaper.

4. The intelligent diaper monitoring device of claim 1, wherein the ionic composition sensor within the sensor array comprises: an ion-selective electrode array configured to detect specific ions within the moisture present in the diaper.

5. The intelligent diaper monitoring device of claim 4, wherein the ion-selective electrode array is configured to detect at least one of sodium, potassium, calcium, and chloride ions.

6. The intelligent diaper monitoring device of claim 1, further comprising: a pressure sensor within the sensor array, configured to detect the position or movements of the wearer.

7. The intelligent diaper monitoring device of claim 1, wherein the wireless communication module comprises: a Bluetooth transceiver for short-range communication with a user device.

8. The intelligent diaper monitoring device of claim 1, further comprising: a Wi-Fi transceiver for long-range communication with a remote server or network.

9. The intelligent diaper monitoring device of claim 1, wherein the housing further comprises: an indicator light configured to provide a visual status of the device's connectivity and operation.

10. The intelligent diaper monitoring device of claim 1, wherein the power source comprises: a rechargeable battery disposed within the housing.

11. The intelligent diaper monitoring device of claim 1, wherein the attachment mechanism is configured to securely attach the housing to the exterior of a diaper.

12. The intelligent diaper monitoring device of claim 1, wherein the housing is waterproof to protect the internal components from moisture exposure.

13. The intelligent diaper monitoring device of claim 1, wherein the physiological sensors within the sensor array further comprise: a pulse oximeter sensor for measuring oxygen saturation levels.

14. The intelligent diaper monitoring device of claim 1, further comprising: a temperature sensor within the sensor array, configured to monitor the ambient temperature within the diaper.

15. The intelligent diaper monitoring device of claim 1, wherein the wireless communication module is configured to transmit data to a dedicated mobile application installed on a user device.

16. The intelligent diaper monitoring device of claim 15, wherein the mobile application is configured to provide real-time alerts and historical data analysis based on the data received from the device.

17. The intelligent diaper monitoring device of claim 1, further comprising: a detachable layer made of soft material, wherein the detachable layer hosts the wetness detection circuit, humidity sensor, and ionic composition sensor, and is attachable to a conventional diaper.
